(19) United States
(12) Patent Application Publication
(10) Pub. No.: US 2025/0226575 A1
(43) Pub. Date: Jul. 10, 2025

(54) LASER DIRECT STRUCTURING ANTENNA ASSEMBLY FOR IN-VIVO DEVICES

(71) Applicant: Given Imaging LTD., Yoqneam (IL)

(72) Inventors: Chen Mann, Kibbutz Merchavia (IL); Baruch Gruman, Lapid (IL); Iddo Diiukman, Zihhron Yaakov (IL)

(21) Appl. No.: 18/853,583

(22) PCT Filed: May 2, 2023
(86) PCT No.: PCT/IL2023/050446

§ 371 Date: Oct. 2, 2024

Related U.S. Application Data
(60) Provisional application No. 63/337,194, filed on May 2, 2022.

(51) Int. Cl. H01Q 1/38; A61B 1/04; H01Q 1/22; H01Q 11/08

(52) U.S. Cl. CPC H01Q 1/38; A61B 1/041; H01Q 1/22; H01Q 11/08

(57) ABSTRACT

A laser direct structuring (LDS) antenna assembly for an in-vivo device includes a carrier and one or more electrical contacts disposed on a first exterior surface of the carrier and configured to electrically couple a PCB to a power source. The LDS antenna assembly also includes a helical antenna having a plurality of loops disposed on and coiled around a second exterior surface of the carrier. The helical antenna includes a first feeding connection configured to connect to a first portion of the PCB and a second feeding connection configured to connect to a second portion of the PCB. The first and second feeding connections are configured to increase an impedance of the helical antenna. An antenna loop embedded on the PCB is configured to operate as one of the plurality of loops of the helical antenna to increase an electrical length of the helical antenna.

### FIELD

This disclosure generally relates to antennas, and more specifically to a helical antenna structure suitable for embedding within compact in-vivo devices.

### BACKGROUND

Capsule endoscopy (CE) allows examining the entire gastrointestinal tract (GIT) endoscopically using a swallowable capsule that captures images as it travels naturally through the GIT. For a typical CE procedure, the capsule, which is about the size of a multi-vitamin, is swallowed by the patient under the supervision of a health professional and the patient is provided with a wearable device, e.g., a sensor belt, a recorder placed in a pouch, and a strap to be placed around the patient's shoulder. The wearable device typically includes a storage device. The capsule captures images as it travels naturally through the GIT. Images and additional data (e.g., metadata) are then transmitted to the recorder that is worn by the patient. The capsule is typically disposable and passes naturally with a bowel movement. The procedure data (e.g., the captured images or a portion of them and additional metadata) is stored on the storage device of the wearable device and downloaded to a computing device for processing.

The capsule may include an imaging device for imaging the GIT, a light source (e.g., one or more light emitting diodes (“LEDs”)) for illuminating the GIT, a power source (e.g., a battery), and a radio frequency (RF) power transmitter coupled with an antenna for transmitting data (e.g., image data) to an external device such as the recorder worn by the patient. Conventional swallowable capsules typically use an antenna that is linearly polarized, meaning that for the orientation of the electric field, polarization is confined to a single plane. Use of such linearly polarized antennas to transmit data from a swallowable capsule is not without drawbacks. For example, as the capsule traverses through the GIT it must undergo many changes in movement direction and spatial (3D) orientation, which may result in a degradation of performance in the communication channel between the capsule and the external device due to the occasional misalignment between the capsule's antenna and a receiving antenna of the external device. However, the small size of the swallowable capsule does not leave much space for a larger antenna, and the capsule's battery may need to power the entire capsule for many hours (e.g., a typical CE procedure may last 7-10 hours). Thus, increasing the capsule's transmission power to reduce communication gaps may result in a shortening of the capsule's operation time (assuming the power consumption of the other components within the capsule remain the same).

To overcome some of the drawbacks of a linearly polarized antenna, some capsule endoscopy systems employ a helical antenna. A helical antenna can be operated in two main radiation modes—normal mode and axial mode. For a very small helical antenna (e.g., a helical antenna with a size that is much smaller than the used radio frequency (“RF”) wavelength (λ)), the maximum RF radiation occurs in the plane perpendicular to the axis of the helical antenna. This mode of operation is referred to as the “normal mode”. In general, the RF radiation field produced by the normal-mode helical antenna is elliptically polarized in all directions though, under particular conditions, the RF radiation field can be circularly polarized. Because of its small size compared to the RF wavelength, the normal-mode helical antenna is generally regarded as having low efficiency and narrow bandwidth. Additionally, in a normal mode helical antenna, it may be difficult to realize a required input impedance for purposes of matching the input impedance with a feeder impedance (e.g., 50 ohms) due to restriction on antenna size.

When the circumference of a helical antenna is near the RF wavelength (λ) of operation, the helical antenna operates in the axial mode. This is a non-resonant traveling wave mode in which instead of standing waves, the current and voltage waves travel in one direction, up the helix. Under particular conditions, a helical antenna operating in the axial mode can also beneficially radiate RF waves with circular polarization along the antenna's axis, off the ends of the antenna. Circularly polarized waves are less vulnerable to multipath fading and have less polarization dependency than linearly polarized waves. However, as opposed to the relatively small normal-mode helical antenna, the relatively large size of axial-mode helical antennas makes it difficult to use axial-mode helical antennas in small devices such as swallowable devices (and other in-vivo devices), implantable devices, etc.

While certain properties of a helical antenna may be beneficial for an in-vivo device or implantable device, there are some drawbacks associated with such antennas that need to be overcome in order for it to be incorporated in implantable devices or in swallowable devices. Thus, it would be beneficial to have an antenna construction that overcomes the aforementioned drawbacks with respect to power consumption, size, and impedance matching.

### SUMMARY

Terms including “generally,” “about,” “substantially,” and the like, as utilized herein, are meant to encompass variations, e.g., manufacturing tolerances, material tolerances, use and environmental tolerances, measurement variations, design variations, and/or other variations, up to and including plus or minus 10 percent.

As used herein, “exemplary” does not necessarily mean “preferred” and may simply refer to an example. As used herein, the term “clinician” refers to any medical professional (e.g., doctor, surgeon, nurse, or the like) or other user involved in operation of the surgical system described herein.

Although the disclosure is not limited in this regard, discussions utilizing terms such as, for example, “processing,” “computing,” “calculating,” “determining,” “establishing,” “analyzing,” “checking,” or the like, may refer to operation(s) and/or process(es) of a computer, a computing platform, a computing system, or other electronic computing device, that manipulates and/or transforms data represented as physical (e.g., electronic) quantities within the computer's registers and/or memories into other data similarly represented as physical quantities within the computer's registers and/or memories or other non-transitory information storage media that may store instructions to perform operations and/or processes.

Provided in accordance with aspects of the present disclosure is a laser direct structuring (LDS) antenna assembly for an in-vivo device. The LDS antenna assembly includes a carrier, a first metal trace and a second metal trace. The first metal trace is disposed on a first exterior surface of the carrier and includes one or more electrical contacts configured to electrically couple to a power source. The second metal trace is disposed separately from the first metal trace on a second exterior surface of the carrier. The second metal trace includes a plurality of loops coiled around the second exterior surface of the carrier to form a helical antenna. The second metal trace also includes first and second feeding connections. The first feeding connection is connected to a first loop of the plurality of loops of the helical antenna and the second feeding connection is connected to a second loop of the plurality of loops of the helical antenna. The first and second feeding connections are configured to electrically couple the helical antenna to a radiofrequency (RF) power transmitter.

In an aspect of the present disclosure, at least a portion of the second metal trace is disposed on an interior surface of the carrier.

In another aspect of the present disclosure, the first and second feeding connections extend from a third exterior surface of the carrier.

In another aspect of the present disclosure, the first and second feeding connections are configured to increase an impedance of the helical antenna for enabling the helical antenna to receive signals from the RF transmitter.

In still another aspect of the present disclosure, the first and second feeding connections are configured to electrically couple to a printed circuit board (PCB) having an antenna loop configured to operate as one of the plurality of loops of the helical antenna.

In yet another aspect of the present disclosure, the antenna loop of the PCB is configured to increase an electrical length of the helical antenna.

In another aspect of the present disclosure, the helical antenna includes a first vertical step and a second vertical step. The first vertical step interconnects the first and second loops of the plurality of loops and is configured to vertically space the first loop from the second loop. The second vertical step interconnects the second loop and a third loop of the plurality of loops to vertically space the second loop from the third loop.

In yet another aspect of the present disclosure, the plurality of loops are parallel to each other and the first and second vertical steps are perpendicular to the plurality of loops.

In an aspect of the present disclosure, the helical antenna includes a via received through the second exterior surface of the carrier. The via is configured to couple the second feeding connection to the second loop of the plurality of loops.

In another aspect of the present disclosure, the first metal trace includes a via received through the first exterior surface of the carrier. The via is configured to electrically couple the one or more electrical contacts to a mounting member for mounting to a PCB.

In still another aspect of the present disclosure, the first and second feeding connections are configured to be soldered to a PCB to electrically couple the helical antenna to the RF power transmitter.

An in-vivo imaging device provided in accordance with the present disclosure includes a PCB, an RF transmitter electrically coupled to the PCB, a power source configured to power the PCB and the RF transmitter, and an LDS antenna assembly electrically coupled to the PCB and the RF transmitter. The LDS antenna assembly includes a carrier, one or more electrical contacts, and a helical antenna. The one or more electrical contacts are disposed on a first exterior surface of the carrier and are configured to electrically couple the power source to the PCB. The helical antenna has a plurality of loops disposed on and coiled around a second exterior surface of the carrier. The helical antenna includes a first feeding connection connected to a first portion of the PCB and a second feeding connection connected to a second portion of the PCB. The first and second feeding connections are configured to increase an impedance of the helical antenna for enabling the RF transmitter to transmit or receive RF signals through the helical antenna. The in-vivo imaging device also includes an antenna loop embedded on the PCB and configured to operate as one of the plurality of loops of the helical antenna to increase an electrical length of the helical antenna.

In an aspect of the present disclosure, the power source includes at least one battery.

In another aspect of the present disclosure, the antenna loop embedded on the PCB includes a first feeding point and a second feeding point. The first feeding point is configured to electrically interconnect the RF transmitter and the first feeding connection of the helical antenna. The second feeding point is configured to electrically interconnect the RF transmitter and the second feeding connection of the helical antenna.

In still another aspect of the present disclosure, the PCB includes a first connection pad and a second connection pad. The first connection pad is electrically coupled to the antenna loop and is configured to be soldered to the first feeding connection of the helical antenna. The second connection pad is electrically coupled to the antenna loop embedded on the PCB and is configured to be soldered to the second feeding connection of the helical antenna.

In still yet another aspect of the present disclosure, the first exterior surface of the carrier includes at least one aperture therethrough configured to enable inspection of a portion of the PCB disposed underneath the LDS antenna assembly.

In another aspect of the present disclosure, the helical antenna includes a via received through the second exterior surface of the carrier and configured to electrically couple the second feeding connection to the plurality of loops.

In still another aspect of the present disclosure, the second feeding connection is electrically coupled to the via by a metallized trace disposed on an interior surface of the carrier.

In still yet another aspect of the present disclosure, the first and second feeding connections extend from a third exterior surface of the carrier.

Another LDS antenna assembly for an in-vivo device provided in accordance with the present disclosure includes a carrier, a PCB, one or more electrical contacts, and a helical antenna. The one or more electrical contacts are disposed on a first exterior surface of the carrier and configured to electrically couple the PCB to a power source. The helical antenna has a plurality of loops disposed on and coiled around a second exterior surface of the carrier. The helical antenna includes a first feeding connection configured to connect to a first portion of the PCB and a second feeding connection configured to connect to a second portion of the PCB. The first and second feeding connections are configured to increase an impedance of the helical antenna. The LDS antenna assembly also includes an antenna loop embedded on the PCB and configured to operate as one of the plurality of loops of the helical antenna to increase an electrical length of the helical antenna.

### BRIEF DESCRIPTION OF THE DRAWINGS

The above and other aspects and features of this disclosure will become more apparent in view of the following detailed description when taken in conjunction with the accompanying drawings wherein like reference numerals identify similar or identical elements.

FIG. 1 shows an in-vivo imaging system according to aspects of this disclosure;

FIGS. 2A and 2B are top, perspective views of an LDS antenna assembly according to aspects of this disclosure;

FIGS. 3A and 3B are side, perspective views of the LDS antenna assembly of FIGS. 2A and 2B according to aspects of this disclosure;

FIGS. 4A and 4B are bottom, perspective views of the LDS antenna assembly of FIGS. 2A and 2B according to aspects of this disclosure;

FIG. 5A is a side, perspective view of a carrier of the LDS antenna assembly of FIGS. 2A and 2B according to aspects of this disclosure;

FIG. 5B is bottom, perspective view of a carrier of the LDS antenna assembly of FIGS. 2A and 2B according to aspects of this disclosure;

FIG. 6A is a side, perspective view of a power source contact of the LDS antenna assembly of FIGS. 2A and 2B according to aspects of this disclosure;

FIG. 6B is a side, perspective view of a helical antenna of the LDS antenna assembly of FIGS. 2A and 2B according to aspects of this disclosure;

FIG. 7A is a side, perspective view of the power source contact, helical antenna, and feeding connections of the LDS antenna assembly of FIGS. 2A and 2B;

FIG. 7B is a side, perspective view of the carrier of the LDS antenna assembly of FIGS. 2A and 2B according to aspects of this disclosure;

FIG. 8A is a side, perspective view of the LDS antenna assembly of FIGS. 2A and 2B coupled to a PCB according to aspects of this disclosure;

FIG. 8B is a side view of the LDS antenna assembly of FIGS. 2A and 2B coupled to the PCB of FIG. 8A and a power source according to aspects of this disclosure;

FIGS. 9A and 9B are bottom, perspective views of the LDS antenna assembly of FIGS. 2A and 2B coupled to antenna traces and connection pads of the PCB of FIGS. 8A and 8B according to aspects of this disclosure;

FIG. 10A is a bottom, perspective view of the helical antenna of the LDS antenna assembly of FIGS. 2A and 2B coupled to the antenna traces and connection pads of FIGS. 9A and 9B according to aspects of this disclosure; and

FIG. 10B is a side, perspective view of the helical antenna of the LDS antenna assembly of FIGS. 2A and 2B coupled to the antenna traces and connection pads of FIGS. 9A and 9B according to aspects of this disclosure.

### DETAILED DESCRIPTION

Provided in accordance with aspects of the present disclosure is a laser direct structuring (“LDS”) antenna assembly for use with an in-vivo device such as, for example, a swallowable capsule of a CE system. The LDS antenna assembly generally includes a three-dimensional (“3D”) carrier and a helical antenna formed from metal traces applied to the carrier by an LDS process, whereby metal traces can be directly transferred onto the 3D surface of an injection-molded structure through the operation of a laser beam. Utilizing an LDS technique makes production processes more efficient and enables an antenna design to be easily mass-produced with lower costs for customer-specific tooling. The LDS technique may include injection molding of thermoplastic materials to form the 3D carrier on which metal traces are deposited through a metallization process. Since adhering metal to a thermoplastic material is difficult, special treatment of the 3D carrier by a laser serves to facilitate the metallization process on the thermoplastic surface. More specifically, by blending a special chemical additive in the thermoplastic materials prior to injection-molding, laser activation can be used to initiate a chemical reaction to form very fine metal particles on the laser-treated surface of the injection-molded 3D carrier such that the deposited metal traces can be properly adhered to the treated surface during the metallization process.

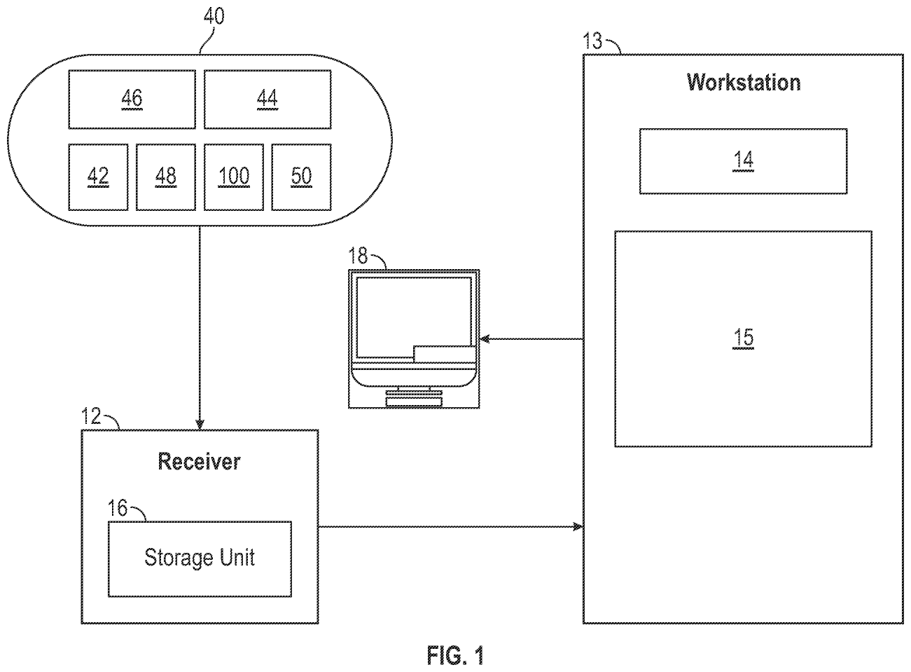

The disclosed LDS antenna assembly is configured to be received and housed within an in-vivo device such as, for example, a swallowable capsule of a CE system (see FIG. 1). The LDS antenna assembly may be coupled to a printed circuit board (PCB) by any suitable method including, but not limited to, surface-mount technology (SMT). For example, the LDS antenna assembly may be a surface-mount device (SMD) mounted directly onto the surface of the PCB. In this regard, the PCB may in some embodiments of this disclosure be considered part of the LDS antenna assembly. The LDS antenna assembly may include a metallized helical antenna disposed on a carrier for transmitting signals from a radiofrequency (RF) power transmitter housed within the in-vivo device and coupled to the PCB. The metallized helical antenna may include a plurality of loops (e.g., three loops) disposed on and coiled around an exterior surface of the carrier. In aspects of this disclosure, at least a portion of the helical antenna may be disposed on an interior surface of a hollow interior of the carrier.

Small or compact in-vivo devices have only a small space for accommodating a communication antenna. Therefore, the size of an antenna that is to be embedded in such small devices has to be on the order of millimeters, and, when a helical antenna is to be used, the number of antenna turns is limited to just a few coil turns (e.g., three or less turns). Therefore, a helical antenna that is designed under the strict space constraints of such a small or compact device may have a very low impedance (e.g., 1-4 ohms). An antenna having such a low impedance may not efficiently interoperate with an RF power transmitter, for example because of increased power loss. To mitigate this problem, the disclosed helical antenna is structured as an autotransformer with two antenna feeding connections, which are connections on the helical antenna by which the RF transmitter transmits (or receives) RF signals. By controlling the locations of the two antenna feeding connections of the helical antenna with respect to the PCB, the two antenna feeding connections serve to step up the impedance of the helical antenna so as to make the helical antenna suitable for operation with an RF power transmitter. In aspects of this disclosure, a trace antenna may be embedded into the PCB to which the LDS antenna assembly is coupled and serve as an additional loop of the helical antenna when the helical antenna is coupled to the PCB, thereby further stepping up the impedance of the helical antenna.

The LDS antenna assembly may also include metallized electrical contacts configured to contact a power source (e.g., a battery) for powering the PCB and/or the LDS antenna assembly. The power source may be used to also power other components within the in-vivo device and/or mounted on the PCB such as, for example, an illumination source, an RF power transmitter, an imaging device, etc.

Those skilled in the art will understand that the disclosed LDS antenna assembly, although described in connection with in-vivo device of a CE system, may also be adapted for use in various small and/or compact devices such as, for example, implants, tools, toys, electronics, or the like.

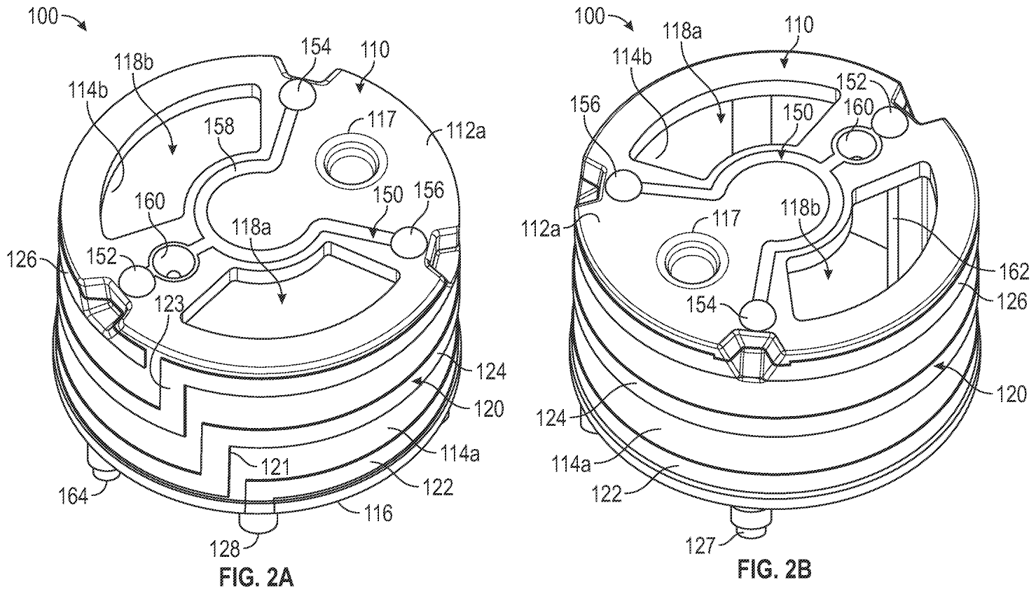

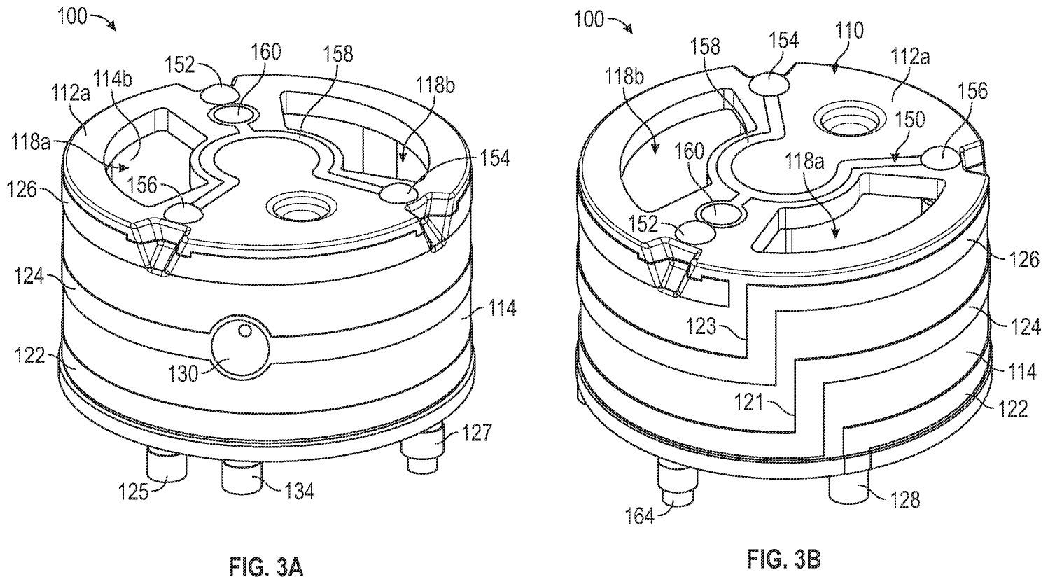

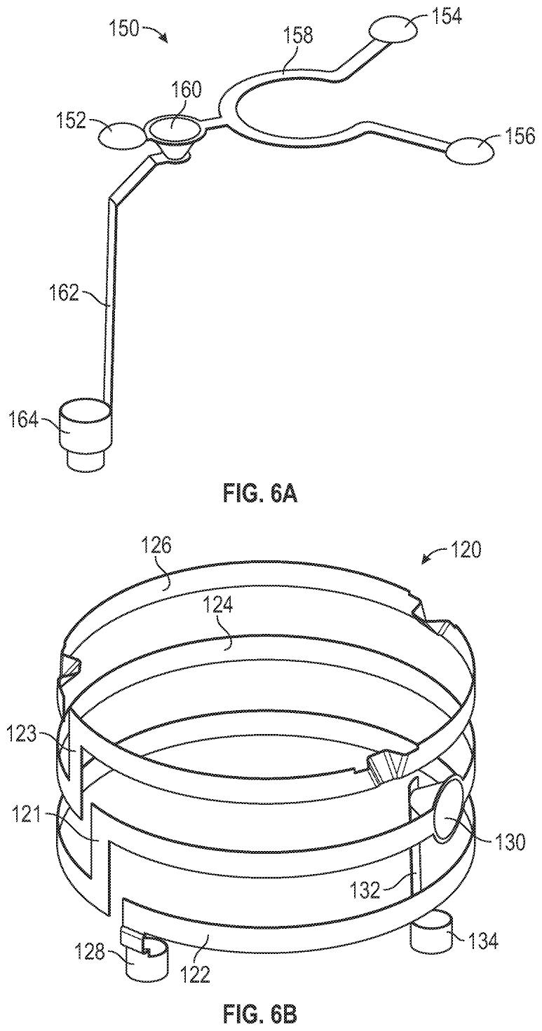

With reference to FIG. 1, a block diagram of an exemplary in-vivo imaging system 10 is shown as an example of a system in which the LDS antenna assembly of the present disclosure may be implemented. In the example shown in FIG. 1, the in-vivo imaging system 10 generally includes an in-vivo imaging device 40, a receiver 12, and a workstation 13. The in-vivo imaging device 40 may be embodied as a swallowable capsule, as shown in the example of FIG. 1, but other suitable implementations may be used. As detailed below with reference to FIGS. 2A-10A, the LDS antenna assembly of this disclosure may be implemented as part of an in-vivo imaging device such as the imaging device 40 and is generally represented throughout the figures by reference number 100. The imaging device 40 includes a power source 48 and a PCB 50. The power source 48 may include one or more batteries or power cells. For example, power source 145 may include silver oxide batteries, lithium batteries, other suitable electrochemical cells having a high energy density, or the like. Other suitable power sources may be used. For example, power source 145 may receive power or energy from an external power source (e.g., an electromagnetic field generator), which may be used to transmit power or energy to the imaging device 40. The imaging device 40 also includes one or more imagers 46 (e.g., two imagers-one on each side of the imaging device 40) for capturing images in a body lumen (e.g., in the GIT), an illumination source (or sources) 42 for illuminating the body lumen, and an RF transmitter 44 that may be positioned anywhere on the PCB 50 and is configured to transmit images (in the form of data frames) captured in-vivo by the imager(s) 46 to the receiver 12 using the LDS antenna assembly 100. In aspects of this disclosure, the RF transmitter 44 may be or include an RF power amplifier.

As detailed below, the power source 48 and the PCB 50 may be electrically and physically coupled to the LDS antenna assembly 100 such that the LDS antenna assembly 100 electrically couples the power source 48 to the PCB 50. This arrangement serves to minimize the number of parts within an in-vivo device in which the LDS antenna assembly 100 is implemented.

The receiver 12 includes a storage unit 16 (e.g., a computer memory, or a hard disk drive) for storing image data received from the imaging device 40 and, in aspects, the receiver 12 may be portable and wearable on the patient's body during receiving (and recording) of the images. The receiver 12 may transfer image data originating from the imaging device 40 to the workstation 13. The workstation 13 may include, or be connected to, an image display device 18 for displaying, among other things, images originating from the imaging device 40.

The workstation 13 may include a data processor 14 and a storage unit 15. The storage unit 15 may serve to store image data received from the receiver 12. The data processor 14 may be configured to execute software instructions stored in the storage unit 15. The data processor 14 may be or include any standard data processor, such as a microprocessor, multiprocessor, accelerator board, or any other serial or parallel high performance data processor. Display device 18 may be a computer screen, a conventional video display, or any other device capable of providing image and/or other data.

During operation, images are captured by the imager(s) 46 and image data representing the images is transmitted by the RF transmitter 44 to the receiver via the LDS antenna assembly 100 using, for example, radiofrequency (RF) waves. The receiver 12 transfers the image data to the workstation 13 for storage, processing, and displaying.

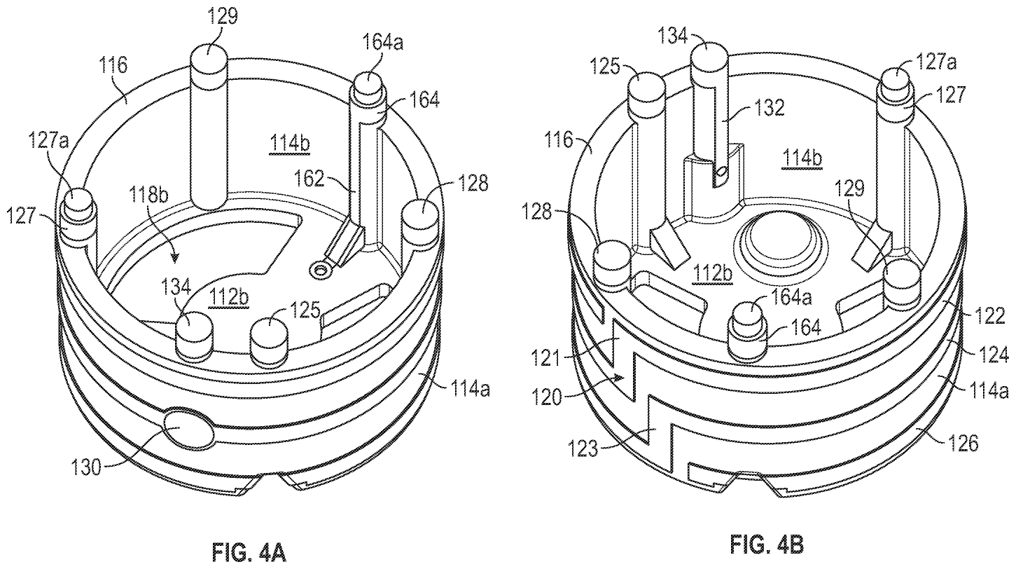

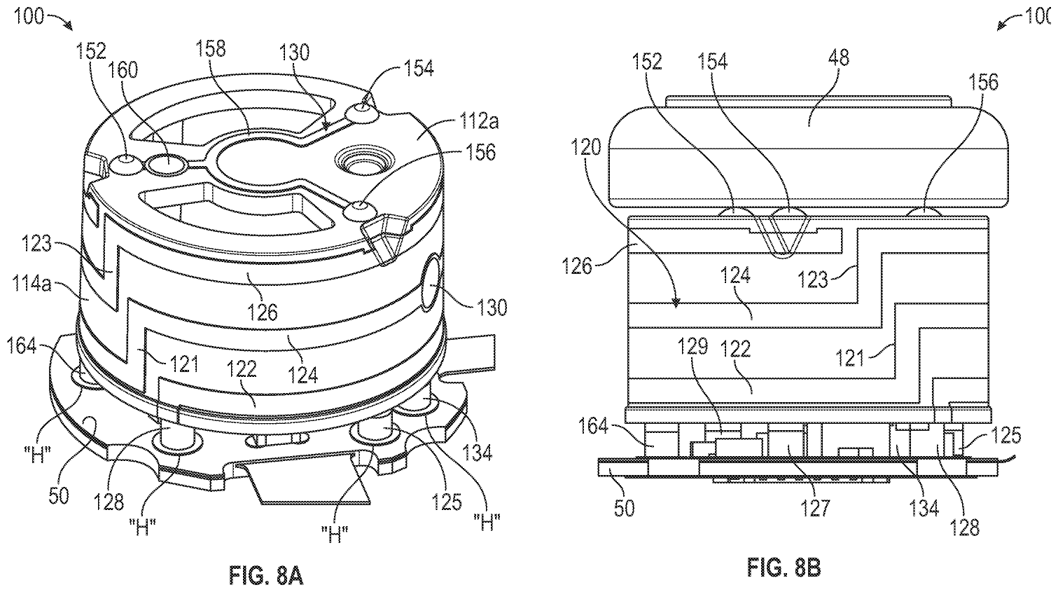

Referring now to FIGS. 2A-7B, the LDS antenna assembly 100 is shown and generally includes a carrier 110 having metal traces disposed thereon. In aspects of this disclosure, the LDS antenna assembly 100 may include a first metal trace forming a power source contact 150 and a second metal trace, separate from the first metal trace, forming a helical antenna 120. The carrier 110 includes a top outer surface 112a, a side outer surface 114a, and a bottom outer periphery surface 116. In the example illustrated throughout the drawings, the carrier 110 is generally cylindrical in shape, however, other suitable shapes (e.g., rectangular) are contemplated. As shown in FIGS. 4A and 4B, the carrier 110 includes a hollow interior having a top inner surface 112b and a side inner surface 114b. The top inner surface 112b is an interior side of the top outer surface 112a and the side inner surface 114b is an interior side of the side outer surface 114a. A pair of apertures 118a and 118b are formed through the top outer surface 112a and the top inner surface 112a of the carrier 110 and serve to allow for visual inspection of the PCB 50 and/or the components disposed on the PCB 50 underneath the carrier 110 once the LDS antenna assembly 100 is secured to the PCB 50 (see FIG. 8A).

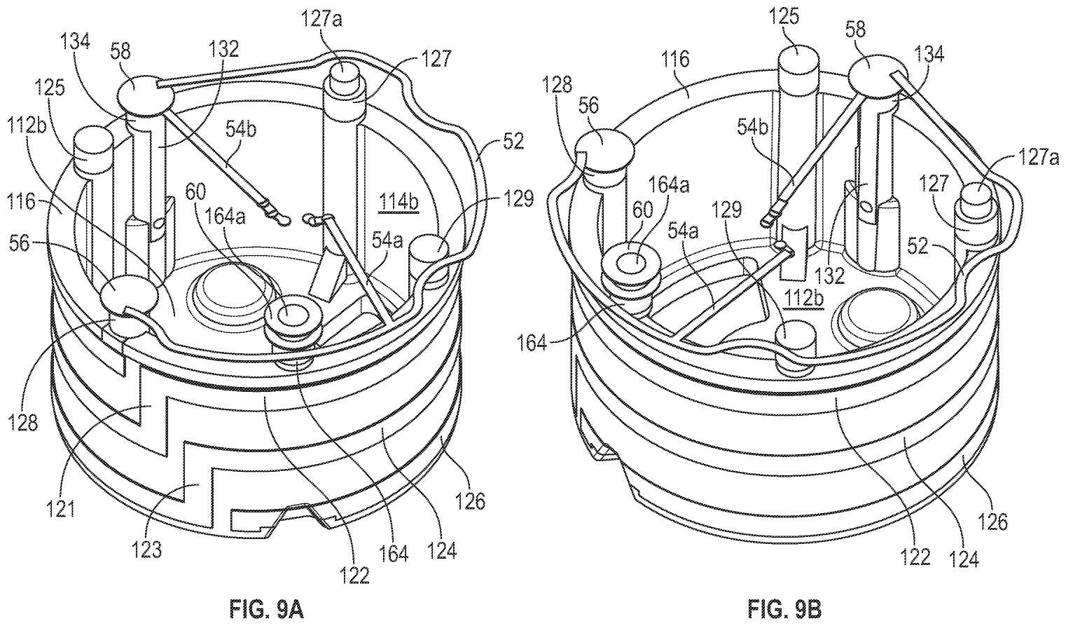

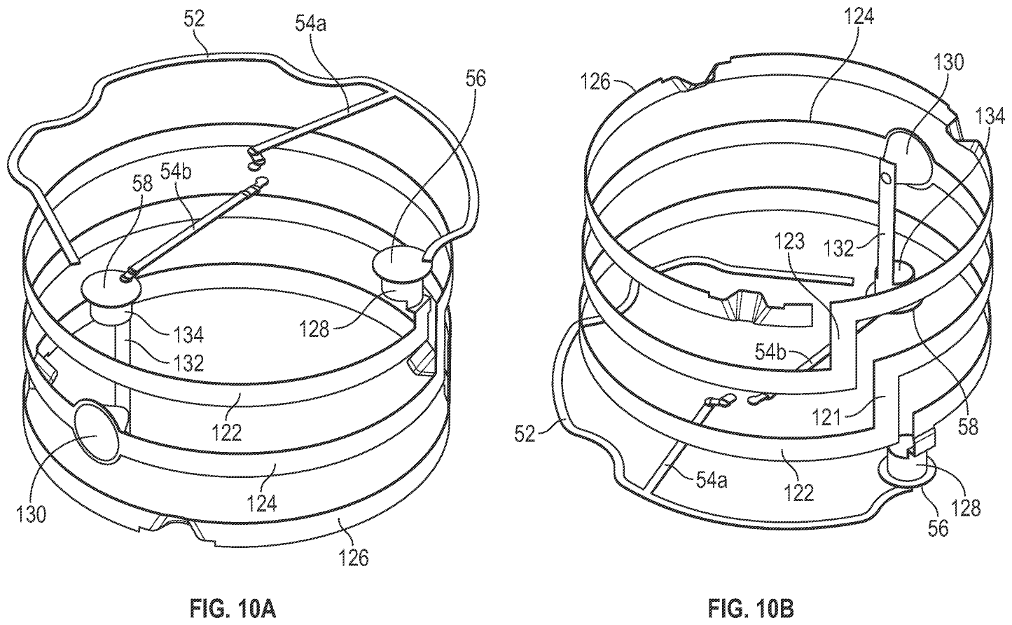

The helical antenna 120 includes a plurality of antenna loops including a first loop 122, a second loop 124, and a third loop 126 disposed on and coiled horizontally around the side outer surface 114a of the carrier 110. The first loop 122 extends from a first mounting member 128 to a first vertical step 121. The first vertical step 121 interconnects and extends perpendicularly between the first loop 122 and the second loop 124 such that the second loop 124 is parallel to and vertically spaced from the first loop 122 around the entirety of the exterior side surface 114a of the carrier 110. The first mounting member 128 is metallized onto a first protrusion 104 extending from the bottom surface 116 of the carrier 110 such that the first mounting member 128 likewise protrudes from the bottom surface 116. The first mounting member 128 may be, for example, a conductive pin configured to be soldered to a connection pad 56 (FIGS. 9A-10B) on the PCB 50. The second loop 124 extends from the first vertical step 121 to a second vertical step 123. The second vertical step 123 interconnects and extends perpendicularly between the second loop 124 and the third loop 126 such that the third loop 126 is parallel to and vertically spaced from the second loop 124 around the entirety of the exterior side surface 114a of the carrier 110. As would be understood, maximizing the vertical spacing between the loops 122, 124, 126 serves to minimize self-capacitance and/or proximity effect losses.

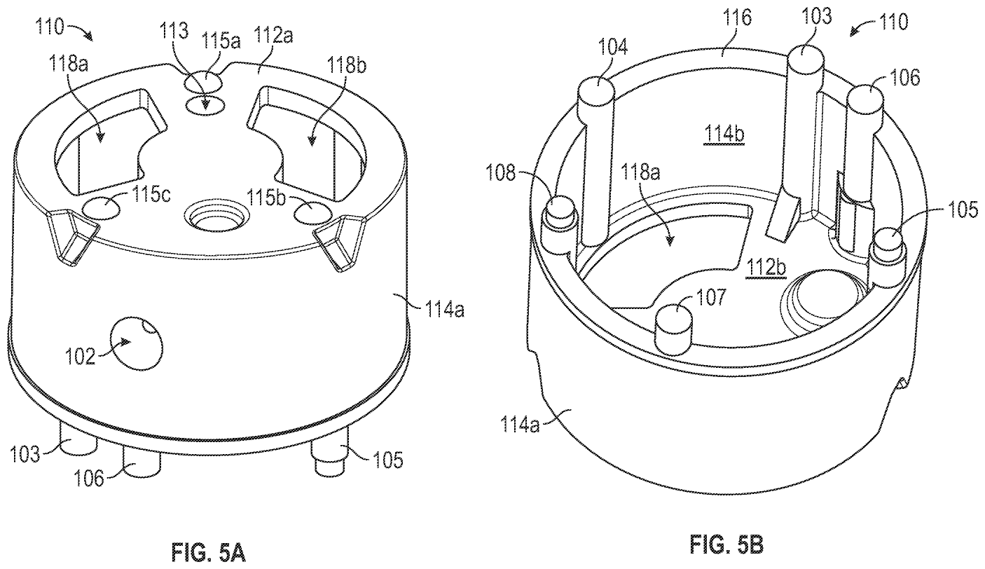

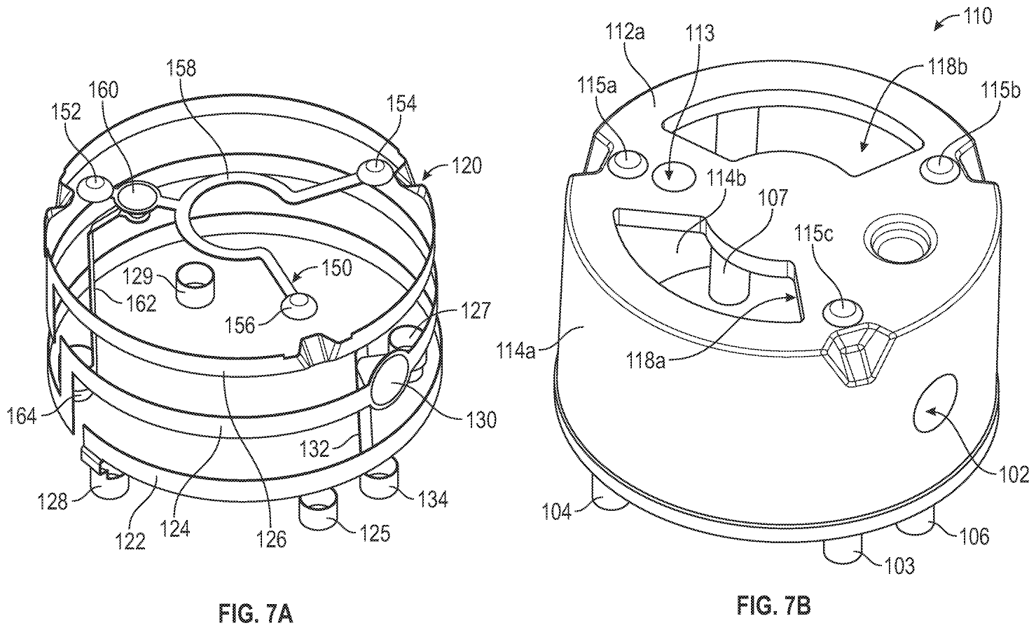

The second loop 124 of the helical antenna 120 includes a via 130 that is received through an aperture 102 (FIGS. 5A and 7B) extending through the exterior and interior side surfaces 114a, 114b of the carrier 110. The via 130 is coupled to a vertically-extending antenna trace 132 disposed on the side interior surface 114b of the carrier 110. The antenna trace 132 interconnects the via 130 to a second mounting member 134. The second mounting member 134 is metallized onto a second protrusion 106 extending from the bottom surface 116 of the carrier 110 such that the second mounting member 134 likewise protrudes from the bottom surface 116. In aspects of this disclosure, the first and second mounting members 128 and 134 may serve as first and second feeding connections, respectively, of the helical antenna 120, for receiving RF power from the RF transmitter 44.

With reference to FIGS. 9A-10B, an additional PCB antenna loop 52 (e.g., a trace antenna) is embedded in the PCB 50 (FIGS. 8A and 8B) and is configured to be coupled to mounting members 128 and 134 of the helical antenna 120 by connection pads 56 and 58, respectively, such that the helical antenna 120 includes a total of four (4) loops (e.g., loops 122, 124, and 126 in addition to the PCB antenna loop 52) to increase the effective electrical length and efficiency of the helical antenna 120, thereby enabling stronger signals from the RF transmitter 44 to be transmitted from the helical antenna 120.

The carrier 110 includes additional protrusions 103, 105, 107 (FIG. 5B) extending from the bottom surface 116 and configured to receive (e.g., by metallization) additional mounting members 125, 127, 129 (FIGS. 4A and 4B) thereon, respectively. The first and second mounting members 128, 134 and the additional mounting members 125, 127, 129 are configured to be received within suitable holes “H” in the PCB 50, examples of which are shown in FIG. 8A, and secured (e.g., via soldering) to connection pads of the PCB 50 to couple the LDS antenna assembly 100 to the PCB 50. In aspects of this disclosure, any one or more of mounting members 125, 127, 128, 129, 134, and 164 may include a specific shape or configuration that is suited to fit only within specific holes “H” in the PCB 50 to dictate the radial orientation of the LDS antenna assembly 100 on the PCB 50 during assembly. This arrangement helps to avoid errors in assembling the LDS antenna assembly 100 on the PCB 50. In the example shown in FIGS. 4A-5B, the mounting members 127 and 164 include an extra protrusion 127a and 164a, respectively, relative to the other mounting members 125, 128, 129, and 134. These extra protrusions 127a and 164a may be formed during metallization due to the corresponding shape of the protrusions 105 and 108 (FIG. 5B), respectively, of the carrier 110 onto which the mounting members 127 and 134 are disposed.

With reference to FIGS. 2A-3B and 6A, the power source contact 150 is disposed on the top exterior surface 112a and includes electrical contacts 152, 154, and 156 interconnected by an intermediate portion 158 disposed between the apertures 118a, 118b. The electrical contacts 152, 154, and 156 are disposed on protrusions 115a, 115b, and 115c (FIG. 5A), respectively, extending from the top exterior surface 112a of the carrier 110 such that the electrical contacts 152, 154, and 156 protrude outward from the top exterior surface 112a to facilitate contact between the power source contact 150 and the power source 48 (FIG. 8B). The arrangement of the intermediate portion 158 and the electrical contacts 152, 154, and 156 branching off from the intermediate portion 158 serves to provide a metallization-free portion 117 of the top exterior surface 112a of the carrier 110 (e.g., the area between the contact points 154 and 156 and partially surrounded by the intermediate portion 158) to allow for handling of the LDS antenna assembly 100 without the need for the handler to contact the metallized helical antenna 120 or the metallized power source contact 150. The power source contact 150 also includes a via 160 received through an aperture 113 extending through the top surfaces 112a, 112b of the carrier 110. The via 160 is interconnected to a third mounting member 164 by a vertically-extending antenna trace 162 disposed on the side interior surface 114b of the carrier 110. The third mounting member 164 is metallized onto a third protrusion 108 extending from the bottom surface 116 of the carrier 110 such that the third mounting member 164 likewise protrudes from the bottom surface 116.

Referring now to FIGS. 8A-10B, the LDS antenna assembly 100 is configured to be coupled to the PCB 50 (FIG. 8A) and to the power source 48 (FIG. 8B). The PCB 50 includes a first connection pad 56 configured to be coupled to the first mounting member 128 and a second connection pad 58 configured to be coupled to the second mounting member 134. The PCB 50 also includes a third connection pad 60 (FIGS. 9A and 9B) configured to be coupled to the third mounting member 164. In aspects of this disclosure, the mounting members 128, 134, 164 may be soldered to the connection pads 56, 58, 60, respectively. The coupling of the first and second mounting members 128, 134 to the connection pads 56, 58 of the PCB 50, respectively, serves to enable electrical communication between the helical antenna 120 and the PCB 50 and/or components (e.g., imagers 46, illumination source 42, RF transmitter 44, etc.) mounted to the PCB 50. The coupling of the third mounting member 164 to the connection pad 60 of the PCB 50 serves to enable delivery of power from the power source 48 to the PCB 50 and/or components (e.g., imagers 46, illumination source 42, RF transmitter 44, etc.) mounted to the PCB 50 when the power source 48 is connected to the electrical contacts 152, 154, 156. The power source 48 may be biased toward the LDS antenna assembly 100 by a suitable mechanical arrangement such as, for example, a spring (e.g., conical spring) disposed within the imaging device 40 and configured to impart a spring force on the power source 48 toward the electrical contacts 152, 154, and 156 to maintain a connection therebetween.

The PCB 50 also includes a first feeding point 54a coupled to the PCB antenna loop 52 embedded in the PCB 50 and a second feeding point 54b electrically coupled to the second mounting member 134 by the connection pad 58. The first and second feeding points 54a, 54b are configured to electrically couple the helical antenna 120 to an integrated circuit (not shown) mounted on the PCB 50 and configured for enabling the helical antenna 120 to transmit signals from the transmitter 44 of the in-vivo imaging device 40 to the receiver 12 (FIG. 1).

Though not explicitly shown, the RF transmitter 44 may be mounted on the PCB 50 and include output terminals (not shown) that are electrically connected to the first and second feeding points 54a, 54b. The first feeding point 54a may be electrically connected to the first mounting member 128 through the connection pad 56 and the second feeding point 54b may be electrically connected to the second mounting member 134 through the connection pad 58. As mentioned above, the first and second mounting members 128 and 134 may serve as first and second feeding connections, respectively, of the helical antenna 120 for receiving RF power from the RF transmitter 44.

As mentioned above, a helical antenna that is designed under the strict space constraints of a compact device such as the imaging device 40 may have a very low impedance (e.g., 1-4 ohms). An antenna having such a low impedance cannot efficiently interoperate with an RF power transmitter (e.g., RF transmitter 44), for example because of increased power loss. To mitigate this problem, the two antenna feeding connections—the first and second mounting members 128 and 134—serve as points on the helical antenna 120 by which the RF transmitter 44 transmits (or receives) RF signals. By electrically coupling the first and second mounting members 128 and 134 to the PCB antenna loop 52 embedded in the PCB 50, the helical antenna effectively includes four loops (e.g., loops 122, 124, 126, and 52) to increase the effective electrical length and efficiency of the helical antenna 120. In this way, the impedance of the helical antenna 120 is further increased, which enables stronger signals from the RF transmitter 44 to be transmitted from the helical antenna 120.

It should be understood that various aspects disclosed herein may be combined in different combinations than the combinations specifically presented hereinabove and in the accompanying drawings. While several aspects of the disclosure have been shown in the drawings, it is not intended that the disclosure be limited thereto, as it is intended that the disclosure be as broad in scope as the art will allow and that the specification be read likewise. Therefore, the above description should not be construed as limiting, but merely as exemplifications of particular aspects. Those skilled in the art will envision other modifications within the scope and spirit of the claims appended hereto.

What is claimed is:

1. A laser direct structuring (LDS) antenna assembly for an in-vivo device, comprising:
a carrier;
a first metal trace disposed on a first exterior surface of the carrier and including at least one electrical contact configured to electrically couple to a power source;
a second metal trace disposed separately from the first metal trace on a second exterior surface of the carrier, the second metal trace including:
a plurality of loops coiled around the second exterior surface of the carrier to form a helical antenna;
a first feeding connection connected to a first loop of the plurality of loops of the helical antenna; and
a second feeding connection connected to a second loop of the plurality of loops of the helical antenna, the first and second feeding connections configured to electrically couple the helical antenna to a radiofrequency (RF) power transmitter.

2. The LDS antenna assembly according to claim 1, wherein at least a portion of the second metal trace is disposed on an interior surface of the carrier.

3. The LDS antenna assembly according to claim 1, wherein the first and second feeding connections extend from a third exterior surface of the carrier.

4. The LDS antenna assembly according to claim 1, wherein the first and second feeding connections are configured to increase an impedance of the helical antenna for enabling the helical antenna to receive signals from the RF transmitter.

5. The LDS antenna assembly according to claim 1, wherein the first and second feeding connections are configured to electrically couple to a printed circuit board (PCB) having an antenna loop configured to operate as one of the plurality of loops of the helical antenna.

6. The LDS antenna assembly according to claim 5, wherein the antenna loop of the PCB is configured to increase an electrical length of the helical antenna.

7. The LDS antenna assembly according to claim 1, wherein the helical antenna includes:
a first vertical step interconnecting the first and second loops of the plurality of loops and configured to vertically space the first loop from the second loop; and
a second vertical step interconnecting the second loop and a third loop of the plurality of loops to vertically space the second loop from the third loop.

8. The LDS antenna assembly according to claim 7, wherein the plurality of loops are parallel to each other and the first and second vertical steps are perpendicular to the plurality of loops.

9. The LDS antenna assembly according to claim 1, wherein the helical antenna includes a via received through the second exterior surface of the carrier and configured to couple the second feeding connection to the second loop of the plurality of loops.

10. The LDS antenna assembly according to claim 1, wherein the first metal trace includes a via received through the first exterior surface of the carrier and configured to electrically couple the at least one electrical contact to a mounting member for mounting to a PCB.

11. The LDS antenna assembly according to claim 1, wherein the first and second feeding connections are configured to be soldered to a PCB to electrically couple the helical antenna to the RF power transmitter.

12. An in-vivo imaging device, comprising:
a printed circuit board (PCB);
a radiofrequency (RF) transmitter electrically coupled to the PCB;
a power source configured to power the PCB and the RF transmitter;
an LDS antenna assembly electrically coupled to the PCB and the RF transmitter, the LDS antenna assembly including:
a carrier;
at least one electrical contact disposed on a first exterior surface of the carrier and configured to electrically couple the power source to the PCB; and
a helical antenna having a plurality of loops disposed on and coiled around a second exterior surface of the carrier, the helical antenna including a first feeding connection connected to a first portion of the PCB and a second feeding connection connected to a second portion of the PCB, wherein the first and second feeding connections are configured to increase an impedance of the helical antenna for enabling the RF transmitter to transmit or receive RF signals through the helical antenna; and
an antenna loop embedded on the PCB and configured to operate as one of the plurality of loops of the helical antenna to increase an electrical length of the helical antenna.

13. The in-vivo imaging device according to claim 12, wherein the power source includes at least one battery.

14. The in-vivo imaging device according to claim 12, wherein the antenna loop embedded on the PCB includes:
a first feeding point configured to electrically interconnect the RF transmitter and the first feeding connection of the helical antenna; and
a second feeding point configured to electrically interconnect the RF transmitter and the second feeding connection of the helical antenna.

15. The in-vivo imaging device according to claim 12, wherein the PCB includes:
a first connection pad electrically coupled to the antenna loop embedded on the PCB, and configured to be soldered to the first feeding connection of the helical antenna; and
a second connection pad electrically coupled to the antenna loop embedded on the PCB, and configured to be soldered to the second feeding connection of the helical antenna.

16. The in-vivo imaging device according to claim 12, wherein the first exterior surface of the carrier includes at least one aperture therethrough configured to enable inspection of a portion of the PCB disposed underneath the LDS antenna assembly.

17. The in-vivo imaging device according to claim 12, wherein the helical antenna includes a via received through the second exterior surface of the carrier and configured to electrically couple the second feeding connection to the plurality of loops.

18. The in-vivo imaging device according to claim 17, wherein the second feeding connection is electrically coupled to the via by a metallized trace disposed on an interior surface of the carrier.

19. The in-vivo imaging device according to claim 12, wherein the first and second feeding connections extend from a third exterior surface of the carrier.

20. A laser direct structuring (LDS) antenna assembly for an in-vivo device, comprising:
a carrier;
a printed circuit board (PCB);
at least one electrical contact disposed on a first exterior surface of the carrier and configured to electrically couple the PCB to a power source; and
a helical antenna having a plurality of loops disposed on and coiled around a second exterior surface of the carrier, the helical antenna including a first feeding connection configured to connect to a first portion of the PCB and a second feeding connection configured to connect to a second portion of the PCB, wherein the first and second feeding connections are configured to increase an impedance of the helical antenna; and
an antenna loop embedded on the PCB and configured to operate as one of the plurality of loops of the helical antenna to increase an electrical length of the helical antenna.

21. The LDS antenna assembly of claim 20, wherein at least a portion of the helical antenna is disposed on an interior surface of the carrier.

22. The LDS antenna assembly of claim 20, wherein the first and second feeding connections extend from a third exterior surface of the carrier.

23. The LDS antenna assembly of claim 20, wherein the first and second feeding connections are configured to increase an impedance of the helical antenna for enabling the helical antenna to receive signals from a RF transmitter connected thereto.

24. The LDS antenna assembly of claim 20, wherein the helical antenna includes:
a first vertical step interconnecting the first and second loops of the plurality of loops and configured to vertically space the first loop from the second loop; and
a second vertical step interconnecting the second loop and a third loop of the plurality of loops to vertically space the second loop from the third loop.

25. The LDS antenna assembly of claim 24, wherein the plurality of loops are parallel to each other and the first and second vertical steps are perpendicular to the plurality of loops.

26. The LDS antenna assembly of claim 20, wherein the helical antenna includes a via received through the second exterior surface of the carrier and configured to couple the second feeding connection to the second loop of the plurality of loops.

27. The LDS antenna assembly of claim 20, wherein the at least one electrical contact includes a via received through the first exterior surface of the carrier and configured to electrically couple the at least one electrical contact to a mounting member for mounting the at least one electrical contact to the PCB.

28. The LDS antenna assembly of claim 20, wherein the first and second feeding connections are soldered to the PCB to electrically couple the helical antenna to a RF power transmitter.
